(19) United States
(12) Patent Application Publication
(10) Pub. No.: US 2007/0275416 A1
(43) Pub. Date: Nov. 29, 2007

(54) Affinity marker for purification of proteins

(75) Inventors: Christian Johannes Gloeckner, Muenchen (DE); Marius Ueffing, Muenchen (DE)

Correspondence Address: CLARK & ELBING LLP, 101 FEDERAL STREET, BOSTON, MA, 02110, US

(73) Assignee: GSF-Forschungszentrum fuer Umwelt und Gesundheit GmbH, Neuherberg (DE)

(21) Appl. No.: 11/803,742

(22) Filed: May 16, 2007

Related U.S. Application Data
(60) Provisional application No. 60/800,917, filed on May 16, 2006.

(51) Int. Cl. G01N 33/53; C07K 16/28

(52) U.S. Cl. 435/7.5; 530/388.22

(57) ABSTRACT

The present invention relates to an affinity marker comprising a FLAG-domain, which contains at least one FLAG-tag, and a Streptavidin-binding domain (Strep-domain), which contains at least two Strep-tags, a protein containing this affinity marker, a nucleic acid which codes for it, a vector or a cell containing the affinity marker, method for the purification of a protein produced in a cell using this affinity marker, and the use of the affinity marker for the purification of a protein produced in a cell.

### CROSS-REFERENCE TO RELATED APPLICATIONS

This application claims the benefit of the filing date of U.S. provisional application 60/800,917, filed May 16, 2006, herein incorporated by reference.

The present invention relates to an affinity marker comprising a FLAG-domain which contains at least one FLAG-tag, and a Streptavidin-binding domain (Strep-domain) which contains at least two Strep-tags, a protein containing this affinity marker, a nucleic acid coding for it and methods for purifying a protein produced in a cell using this affinity marker.

In many areas of industry and research, nowadays pure or even ultrapure products are required, since product quality is often determined by their purity. This applies in particular to proteins produced by biotechnological methods, as these are now finding increasing application in a number of industrial products and processes. They are used for example as medicinal products, for diagnostic or scientific purposes. Furthermore, they are used in many areas of everyday life, for example as diet supplements, as additives in the food industry, for example as baking aids or in cheese-making, but also for example in papermaking, in the hygiene area or in detergents. Ultrapure proteins are in addition required for analytical methods, e.g. for elucidation of structure.

Often it is necessary for biotechnologically produced proteins from eukaryotic cells to be further purified, in order to obtain the product in the desired purity. Therefore there is a growing demand for suitable purification techniques. When producing products by means of biotechnological methods, generally cells are altered by genetic engineering so that they produce the protein of interest. These cells are usually grown in complex cell culture media, which contain sources of nutrients and for example growth factors. Therefore it is necessary to separate the protein of interest both from the constituents of the cell culture medium and from the other cellular constituents of the (eukaryotic) cells that are used for production of the protein. At the same time it is desirable that the aids used for purification should have as little adverse effect as possible on the production of the protein in the cells.

There are already many known methods by which products can be separated from other constituents. As a rule these make use of the differences in physical and chemical properties of the constituents of a sample that is to be purified. Conventional methods of purification and isolation include for example extraction, precipitation, recrystallization, filtration, centrifugation, washing and drying. The separation techniques and principles of adsorption, chromatography or ion exchange are also used. Various column materials are available for chromatographic methods, and make it possible to adapt the purification process to the particular product that is to be purified, making use of the differences in migration rates of the individual constituents, based for example on charge or hydrophobicity.

Affinity chromatography, in which a product, as a rule a protein, is separated and thus purified on the basis of its affinity for a binding partner, is especially suitable. So-called tags have now been developed, which are for example attached to a protein that is to be purified. The tag possesses an affinity for a particular binding partner; this can be utilized in affinity chromatography. Such tags are in principle of universal application and can be attached to various molecules. In this way it is possible to purify different molecules, especially proteins, with the same method of purification. Thus, ideally, the method does not need to be adapted specially to the particular product.

Especially for analyzing and purifying proteins, the technique of affinity labeling, i.e. the attachment of a marker or tag to a protein by techniques of molecular biology, is now a frequently used method. In this technique, the primary sequence of any protein is expanded by just a few amino acids by means of recombinant techniques. The presence of a specific binding molecule with high affinity, e.g. an antibody, with a known recognition sequence, is decisive.

A number of (affinity) tags or (affinity) markers are known at present. These are usually divided into 3 classes according to their size: small tags have a maximum of 12 amino acids, medium-sized ones have a maximum of 60 and large ones have more than 60. The small tags include the Arg-tag, the His-tag, the Strep-tag, the Flag-tag, the T7-tag, the V5-peptide-tag and the c-Myc-tag, the medium-sized ones include the S-tag, the HAT-tag, the calmodulin-binding peptide, the chitin-binding peptide and some cellulose-binding domains. The latter can contain up to 189 amino acids and are then regarded, like the GST-and MBP-tag, as large affinity tags.

In order to produce especially pure proteins, so-called double tags or tandem tags were developed. In this case the proteins are purified in two separate chromatography steps, in each case utilizing the affinity of a first and then of a second tag. Examples of such double or tandem tags are the GST-His-tag (glutathione-S-transferase fused to a polyhistidine-tag), the 6×His-Strep-tag (6 histidine residues fused to a Strep-tag (see below)), the 6×His-tag100-tag (6 histidine residues fused to a 12-amino-acid protein of mammalian MAP-kinase 2), 8×His-HA-tag (8 histidine residues fused to a haemagglutinin-epitope-tag), His-MBP (His-tag fused to a maltose-binding protein, FLAG-HA-tag (FLAG-tag (see below) fused to a haemagglutinin-epitope-tag), and the FLAG-Strep-tag (see below).

Some of these double tags were developed for the purification of proteins from prokaryotic cells. For example, a Flag-Strep II-tag was used for purification of HynL-HybC₂ complex from bacteria, which consisted of a Flag-tag and a Strep II-tag (Fodor et al., 2004, Appl Environ Microbiol., 70: 712-721).

Often, however, it is necessary or desirable to purify proteins that were expressed by eukaryotic cells, e.g. if the protein is modified posttranslationally or proteins that are additionally present in the cell, and which bind to the target protein, are to be purified at the same time. The complexity of eukaryotic proteomes makes the purification of proteins that are expressed by eukaryotes challenging, and as a rule means that an individual purification protocol must be established for each protein. As a rule, therefore, the aforementioned double tags cannot be applied directly in eukaryotic systems.

However, double tags have already been developed for purification of proteins from eukaryotic cells as well. An example of such an expression system is disclosed in WO 00/09716. In this method, biomolecule and/or protein complexes are fused with two different affinity tags, one of which contains an IgG-binding domain of staphylococcus protein A. In practice, such a TAP system (Tandem Affinity Purification system) has been established for yeasts, consisting of a combination of a calmodulin-binding peptide and a protein A tag, with a cleavage site for TEV-protease (protease from the “Tobacco Etch Virus”) between the two components.

The double tag system available at present for eukaryotes, the TAP-system, has essentially three drawbacks:

1. Size

The size of the TAP-tag described above is approx. 21 kDa. The larger the tag, the greater is the probability of the tag impairing the function of the molecule to be purified. Therefore tags that are as small as possible are generally preferred. Moreover, there is a danger of the tag being cleaved or digested proteolytically by the target molecule.
   2. Possible Interferences
Interactions frequently occur between the tag components and the cellular proteins in the mammalian cellular system during expression of the labeled target molecule in a cell. During expression in cells of higher organisms, especially animals, the calmodulin-binding peptide can enter into interactions with other calcium-binding proteins. Owing to the interaction with other proteins, calmodulin can no longer bind to the binding partner, so that purification is disturbed.
   3. Dependence on Calcium Concentration
A defined calcium concentration is required for the calmodulin-binding peptide to bind to the calmodulin in the carrier material. Many buffer systems, especially for cell cultures, use calcium scavengers, for example EDTA or EGTA. These constituents present in buffers can also have an adverse effect on purification.

In addition, double tags from other tags have also been described recently for use for higher eukaryotes.

Thus, Yang and coworkers (Yang et al., 2006, Proteomics 6: 927-935) compared various double tags. In the Drosophila cell culture system, purification of USP and dHNF4 associated protein complexes was investigated using, on the one hand, the original TAP system (calmodulin-binding peptide, protein A tag, cleavage site for TEV-protease, see above) and on the other hand double tags from a combination of 3×Flag- and 6×His-tags, and the efficiencies of the two tag combinations were compared in the purification of various proteins. Whereas the combination of 3×Flag- and 6×His-tag gave an efficiency from 10.6% to 18.6%, with the TAP system it was only possible to achieve yields of less than 1%.

In another experimental setup, the properties of various tags, e.g. HIS, CBP, CYD (covalent yet dissociable NorpD peptide), Strep II, FLAG, HPC (heavy chain of protein C), GST and MBP were compared with respect to their purification properties in various systems (Lichty et al., 2005, Protein Expr Purif 41: 98-105). The authors recommend, taking into account their results, a combination of 6×His- and Strep II-tag for double purification.

The problem to be solved by the present invention was accordingly to provide a further affinity marker, which is suitable for the purification of proteins especially from eukaryotic cells and does not have the aforementioned drawbacks. In particular, a problem to be solved by the present invention was to provide an affinity marker for the purification of proteins from eukaryotic cells, offering maximum possible yields with preferably highest possible purity of the purified protein.

This problem was solved with an affinity marker containing a FLAG-domain which contains at least one FLAG-tag, and a Streptavidin-binding domain (Strep-domain) which contains at least two Strep-tags.

Thus, a first object of the present invention is an affinity marker containing a FLAG-domain which contains at least one FLAG-tag, and a Streptavidin-binding domain (Strep-domain) which contains at least two Strep-tags.

The affinity marker according to the invention displayed a surprisingly high yield in the purification of proteins of eukaryotic expression systems and provided, for example for the purification of B-Raf from HEK293 cells, an efficiency of about 70%. Moreover, purification using the affinity marker according to the invention also produced an especially pure protein. Purities of >97%, especially >99% of the desired protein(s) were achieved in various test systems.

The inventors found that, especially for use in purification from eukaryotic cells, especially mammalian cells, a Strep II-tag is not adequate for achieving satisfactory efficiency in the mammalian cell culture system. Therefore, a Strep-domain with two Strep-tags was used. Surprisingly, the affinity marker containing a FLAG-domain that contains at least one FLAG-tag, and a Streptavidin-binding domain (Strep-domain) that contains at least two Strep-tags, in this system, satisfies the requirements on purity and yield and thus represents a definite improvement over the tags previously available.

An affinity marker is a molecule that has domains which can bind with high affinity to specific binding partners. An affinity marker according to the invention has a FLAG-domain and a Strep-domain. These domains each possess a high affinity for a suitable matrix, which is covered with the specific binding partner. In the case of the FLAG-domain, the principle of separation is based on the specific binding between the FLAG-tag(s) and e.g. a specific anti-FLAG antibody. In the case of the Strep-domain, the principle of separation is based on the specific binding between the Strep-tag(s) and e.g. Streptavidin, Strep-tactin or a specific antibody.

The FLAG-domain contains a FLAG-tag. The FLAG-tag used at first was a leader peptide comprising 11 amino acids of the Gene-10 product from the bacteriophage T7, which was then, for purification of GAL 4 (yeast transcription factor), fused to its amino terminus.

The FLAG-tag according to the present invention is an amino-acid-based marker, as described for example in EP 0 150 126, U.S. Pat. No. 4,703,004, U.S. Pat. No. 4,782,137 and U.S. Pat. No. 4,8151,341 and which in particular contains or consists of the sequence DYK, preferably the sequence DYKD (SEQ ID NO: 1). In addition to these sequences, other amino acids can be present, preferably hydrophilic amino acids for example R (Arg), D (Asp), E (Glu) and K (Lys) and/or amino acids with aromatic side chains for example Y (Tyr), F (Phe), H (His) and W (Trp). Examples of such FLAG-tags are disclosed in the aforementioned patent specifications and can be used within the scope of the present invention.

A preferred FLAG-tag contains or consists of the sequence DYKDDDDK (SEQ ID NO: 2), MDYKDDDDK (SEQ ID NO: 3), DFKDDDK (SEQ ID NO: 4), DYKAFDNL (SEQ ID NO: 5), DYKDHDG (SEQ ID NO: 6), MDFKDDDDK (SEQ ID NO: 7), MDYKAFDNL (SEQ ID NO: 8), DYKDHDI (SEQ ID NO: 9), DYKDH (SEQ ID NO: 10), DYKDD (SEQ ID NO: 11), DYKDHD (SEQ ID NO: 12) and/or DYKDDD (SEQ ID NO: 13). The most preferred sequence is DYKDDDDK, especially for a FLAG-domain with only one FLAG-tag.

A number of monoclonal antibodies against these tags have been described and are commercially available (e.g. from Sigma-Aldrich Chemie GmbH, Munich, Germany), and as a rule the monoclonal antibodies M1, M2 and M5 are used. The individual FLAG-tags can then have different affinities for the various antibodies. Thus, the octapeptide DYKDDDDK and the shorter peptide DYKD are recognized with almost equal affinity by M1 (Knappik et al., 1994, Biotechniques 17: 754-761). Furthermore, for example, the peptide MDFKDDDDK is bound by M5 and MDYKAFDNL by M2 (Slootstra et al., 1997, Mol Divers 2: 156-164).

The term FLAG-tag also covers modified FLAG-tags, which were derived from the FLAG-tags described above, especially the tag with the sequence DYKDDDDK, by amino acid insertion, deletion or substitution.

Preferably, a modified FLAG-tag is a FLAG-tag according to the present invention, if the affinity of an antibody for the modified tag is at most 100-times, preferably at most 50-times, more preferably at most 30-times and even more preferably at most 10-times lower than for the unmodified tag.

Usually the antibodies have an affinity for the epitope of approx. 10⁷ to 10⁸ (mol/l)⁻¹. However, antibodies with affinities of up to 10¹⁰ (mol/l)⁻¹ have also been described. For example, an affinity of 1.5×10⁸ was determined for a monoclonal M2 antibody to the FLAG-tag commercially available from Sigma (Wegner et al., 2002, Anal. Chem. 74: 5161-5168). Therefore the affinity of an antibody specific to the epitope or modified epitope is preferably at least approx. 10⁵ (mol/l)⁻¹, more preferably at least approx. 10⁶ (mol/l)⁻¹, even more preferably at least approx. 10⁷ (mol/l)⁻¹, yet more preferably at least approx. 10⁸ (mol/l)⁻¹ and most preferably at least approx. 10⁹ (mol/l)⁻¹. Methods for the determination of affinity constants and rate constants of antibodies are well known in the prior art. For example, this can be done with biospecific interaction analysis using BIAcore (Pharmacia Biosensor). The method is described for example in DE19643314.

If amino acid substitutions are carried out, conservative amino acid substitutions are preferred. In conservative amino acid exchanges, amino acids of the same kind are exchanged, e.g. a basic amino acid for another basic amino acid (K, R, H), an amino acid with an aromatic side chain for another amino acid with an aromatic side chain (F, Y, W) or an amino acid with an aliphatic side chain for another with an aliphatic side chain (G, A, V, L, I).

In a preferred embodiment, the sequence of the modified epitope has at least 65%, more preferably at least 75% and most preferably at least 85% sequence identity with the epitope as defined above, especially the epitope DYKDDDDK.

In another preferred embodiment, at most 3, more preferably at most 2 and most preferably at most 1 amino acid(s) are modified by insertion, deletion or substitution, especially in the sequence DYKDDDDK.

In a further preferred embodiment, the FLAG-domain contains not just 1 FLAG-tag, but at least 2 or 3 FLAG-tags. The individual FLAG-tags are as defined above. They may be identical or different. Preferably there are at least 1, 2 or 3 FLAG-tags, which contain or consist of the sequence DYK, especially DYKD, more preferably DYKDH or DYKDD and most preferably DYKDHD or DYKDDD.

If the FLAG-domain possesses more than 1 FLAG-tag, the individual FLAG-tags can follow one another directly or can be joined by a linker (FLAG-linker). The linker can be any suitable linker, especially a linker of amino acid residues.

Preferred FLAG-linkers are short peptide linkers consisting of at most 10, more preferably at most 8, even more preferably at most 6, 5, 4, 3 or 2 and most preferably at most 1 amino acid residue. Preferably each of these amino acid residues is an asparagine, lysine, histidine or leucine residue. An especially preferred linker contains or consists of one of the sequences HDI, HDG or DDDK.

Even more preferably, the FLAG-domain contains or consists of several, especially 3 FLAG-tags, especially with 3 FLAG-tags which each begin with the sequence DYKD and comprise a total of 6, 7, 8, 9 or 10, especially 7 or 8 amino acid residues. Preferred sequences for these FLAG-tags are DYKDHDG, DYKDHDI and DYKDDDDK. Even more preferably the FLAG-domain contains or consists of the sequence DYKDHDGDYKDHDIDYKDDDDK (SEQ ID No.: 14).

A further constituent of the affinity marker is the Strep-domain. The Strep-domain contains at least 2 Strep-tags. In one embodiment the Strep-domain contains at least 3, 4, 5 or 6 Strep-tags. If the Strep-domain contains 2 Strep-tags it is a di-Strep-tag, if it contains more than 2 Strep-tags it is a multi-Strep-tag. The di- or multi-Strep-tags are capable in particular of cooperative binding to in each case a single Streptavidin tetramer or Streptavidin dimer. Cooperative binding leads to stronger binding of the Strep-domain to a Streptavidin-receptor. The di- and multi-Strep-tags based on Streptavidin are described in more detail in DE10113776.

Each Strep-tag contains at least the sequence histidine-proline-Xaa-, where Xaa represents either glutamine, asparagine or methionine. The Strep-tags consequently contain the sequences histidine-proline-glutamine, histidine-proline-asparagine and/or histidine-proline-methionine.

A preferred Strep-tag is a Strep-tag of the sequence WSHPQFEK (SEQ ID No. 15) or a derivative thereof. A derivative is obtained by amino acid insertion, deletion and/or substitution, these being defined as described above for the FLAG-domain. Preferably a derivative of the Strep-tag is a Strep-tag according to the present invention, if the affinity of a binding partner for the derivative is at most 100-times, preferably at most 50-times, more preferably at most 30-times and even more preferably at most 10-times lower than for the unmodified Strep-tag of the sequence WSHPQFEK.

Usually each Strep-tag has an affinity (K_{D}) from approx. 10⁻⁵ to approx. 10⁻⁶ mol/l for Streptavidin. For example, for the Strep-tag II when using wild-type Streptavidin or variants thereof, affinities (K_{D}; mol/l) from 1.3×10⁻⁵ to 1.0×10⁻⁶ were determined (Voss and Skerra, 1997, Protein Eng. 10: 975-982). Therefore the affinity of a Strep-tag for a specific binding partner is preferably at least approx. 10⁻³ mol/l, more preferably at least approx. 10⁻⁴ mol/l, even more preferably at least approx. 10⁻⁵ mol/l, still more preferably at least approx. 10⁻⁶ mol/l and most preferably at least approx. 10⁻⁷ mol/l. The affinity of the derivatives can be determined on the basis of the test described in DE19641876 (see Example 5 there).

If amino acid substitutions are carried out, conservative amino acid substitutions (as defined above) are preferred. In the case of amino acid deletions, one or more amino acids are removed, preferably at the C- or N-terminal end of the sequence. In the case of amino acid additions, additional amino acids are inserted. Preferably these are amino acids that do not differ substantially from the surrounding amino acids with respect to e.g. size and charge, i.e. amino acids of the same type as defined previously for conservative amino acid exchanges.

The sequence of the derivative is then at least 62%, more preferably at least 75%, even more preferably at least 85% identical to the sequence WSHPQFEK.

In another preferred embodiment, at most 3, more preferably at most 2 and most preferably at most 1 amino acid(s) are modified by insertion, deletion or substitution.

The 2 or more Strep-tags of the Strep-domain can follow one another directly or can be joined via a linker, the Strep-linker. The linker can be any suitable linker, especially a linker comprising amino acid residues.

In a preferred embodiment the linker is a linker consisting of an amino acid chain, and the linker can contain any arbitrary amino acid. In a further preferred embodiment the Strep-linker consists of 1, 2, 3, 4, 5, 6, 7, 8, 9, 10, 11, 12, 13, 14, 15, 16, 17, 18, 19 or at most 20 amino acid residues (see also DE10113776). Preferred amino acid residues are those which do not hamper the binding of the Strep-tag and the respective binding partner, e.g. Streptavidin. Therefore small and/or uncharged amino acid residues are preferred. Examples of suitable amino acids are glycine and serine. Glycine-based Strep-linkers are especially preferred, i.e. linkers consisting of glycine to at least 30%, preferably at least 50%, more preferably at least 75%, even more preferably at least 90% and most preferably at least 100%. Linkers of amino acid chains comprising glycine and serine residues or glycine and alanine residues to at least 80% or even to 100% are especially preferred. These glycine-based Strep-linkers are even more preferred if they consist of at least 8, 10 or 12 amino acid residues. The Strep-linkers which contain or consist of the sequences (G)₈, (G)₁₂, GAGA, (GAGA)₂, (GAGA)₃, (GGGS)₂ and/or (GGGS)₃ are even still more preferred.

In a preferred embodiment the Strep-domain contains 2, 3 or 4 Strep-tags, especially preferably 2 or 4 Strep-tags and most preferably 2 Strep-tags, preferably Strep-tags of the sequence WSHPQFEK.

Strongly preferred Strep-domains contain or consist of the following sequences WSHPQFEK-(X)ₙ-WSHPQFEK (SEQ ID No.: 16), where X represents an arbitrary amino acid and n is an integer from 5 to 20, especially 8 to 12, more preferably 8, 10 or 12 and most preferably 8 or 12 (SEQ ID Nos.: 17-21). The sequence when X is glycine or serine, especially glycine, is even more preferred. Examples of this are the sequences WSHPQFEK-G₈-WSHPQFEK (SEQ ID No.: 22) and WSHPQFEK-G₁₂-WSHPQFEK (SEQ ID No.: 11). A Strep-domain which contains or consists of the sequence WSHPQFEK-(GGGS)ₙ-WSHPQFEK, where n is 1, 2, 3, 4 or 5, preferably 2 or 3, is also strongly preferred (SEQ ID NOs.: 23-28).

In the affinity marker according to the present invention, each FLAG-domain as defined above can be combined with each Strep-domain as defined above. Affinity markers in which the Strep-domain contains two Strep-tags, especially 2 Strep-tags with the amino acid sequence WSHPQFEK, which are joined together e.g. by a glycine-spacer of 8 or 12 amino acids, and in which the FLAG-domain contains 1 or 3 FLAG-tags, especially the sequences DYKDDDDK or DYKDHDGDYKDHDIDYKDDDDK, are especially preferred.

The FLAG-domain and the Strep-domain can be joined together in the affinity marker in various ways. On the one hand the two domains can be linked e.g. through interactions on the basis of different electron density distributions, for example on the basis of hydrogen bridge bonds, van der Waals forces and/or dipole-dipole interactions. Preferably it can also be covalent binding.

In the case of covalent binding, the two domains can either be joined directly, or via a spacer. The spacer can have any suitable structure, but it is preferably an amino acid chain, especially one with less than 50, preferably less than 25, more preferably less than 20 and even more preferably less than 15 amino acids. If no cleavage sites for an enzyme (see below) are provided in the spacer, the spacer can also consist of 10, 9, 8, 7, 6, 5, 4, 3, 2 or 1, preferably 5 or fewer, amino acids. The arrangement in the affinity marker can be either a Strep-domain-spacer-FLAG-domain or a FLAG-domain-spacer-Strep-domain.

If it is necessary or desirable to split off at least one of the two domains, the affinity marker contains at least one cleavable linkage between the FLAG-domain and the Strep-domain.

A cleavable linkage means any joining of the two domains that permits the two domains to be separated by suitable means. In the case of, for example, an interaction based on different electron density distributions or an ionic bond, the linkage between the two domains can be separated for example by altering the environment, for example by changing the pH, by altering the ionic strength or the like. If the two domains are bound together covalently, the covalent linkage can, for example, be an easily splittable covalent bond, which can easily be split e.g. by adding acid or base or other chemicals such as certain catalysts, without impairing the two domains or other combinations that are associated with the present invention.

In one embodiment, this cleavable linkage can be a cleavage site, for example a cleavage site for an enzyme. This can be located, for example, in the spacer. The cleavage site could for example be a protease cleavage site. Examples of proteases are chymotrypsin, trypsin, elastase, and plasmin; the corresponding cleavage sites are known to a person skilled in the art. Since the molecule to be purified is a protein, specific proteases, especially proteases from viruses that normally attack plants, are preferred. Examples of suitable specific proteases are thrombin, factor Xa, Igase, TEV-protease from the “Tobacco Etch Virus”, the protease PreScission (Human Rhinovirus 3C Protease), enterokinase or Kex2. TEV-protease and PreScission are especially preferred.

Examples for the structure of suitable affinity markers are the following structures; in said examples, the order of the elements (from N- to C-terminal) can be read from left to right or from right to left:

Examples of affinity markers with 2 Strep-tags and one FLAG-tag:

WSHPQFEK-Strep-Linker-WSHPQFEK-Spacer- DYKDXXXX
WSHPQFEK-G₈-WSHPQFEK-Spacer- DYKDXXXX
WSHPQFEK-G₁₂-WSHPQFEK-Spacer- DYKDXXXX
WSHPQFEK-(GGGS)₂-WSHPQFEK-Spacer- DYKDXXXX
WSHPQFEK-(GGGS)₃-WSHPQFEK-Spacer- DYKDXXXX

where X can be any amino acid selected from Y, F, H, W, R, D, E and K, especially D, H and K. XXXX is preferably equal to DDDK (SEQ ID NO: 29).

Examples of affinity markers with 2 Strep-tags and 3 FLAG-tags, where the FLAG-linkers 1 and 2 can be identical or different:

WSHPQFEK-Strep-Linker-WSHPQFEK-Spacer-DYKD-FLAG-
Linker 1--DYKD-FLAG-Linker 2-DYKDXXXX
WSHPQFEK-G₈-WSHPQFEK-Spacer-DYKD-FLAG-Linker 1-
DYKD-FLAG-Linker 2-DYKDXXXX
WSHPQFEK-G₁₂-WSHPQFEK-Spacer-DYKD-FLAG-Linker 1-
DYKD-FLAG-Linker 2-DYKDXXXX
WSHPQFEK-(GGGS)₂-WSHPQFEK-Spacer-DYKD-FLAG-Linker
1-DYKD-FLAG-Linker 2-DYKDXXXX
WSHPQFEK-(GGGS)₃-WSHPQFEK-Spacer-DYKD-FLAG-Linker
1-DYKD-FLAG-Linker 2-DYKDXXXX
WSHPQFEK-Strep-Linker-WSHPQFEK-Spacer-
DYKDHDGDYKDHDIDYKDDDDK
WSHPQFEK-G₈-WSHPQFEK-Spacer-
DYKDHDGDYKDHDIDYKDDDDK
WSHPQFEK-G₁₂-WSHPQFEK-Spacer-
DYKDHDGDYKDHDIDYKDDDDK
WSHPQFEK-(GGGS)₂-WSHPQFEK-Spacer-
DYKDHDGDYKDHDIDYKDDDDK
WSHPQFEK-(GGGS)₃-WSHPQFEK-Spacer-
DYKDHDGDYKDHDIDYKDDDDK

where the FLAG-linker 1 and the FLAG-linker 2 preferably consist of 3 amino acid residues, more preferably selected from D, H, E, G, I and K, especially D, H, I and K, and where X can be any amino acid selected from Y, F, H, W, R, D, E and K, especially D, H and K. XXXX is preferably equal to DDDK (SEQ ID NO: 29).

The most preferred affinity markers contain or consist of the sequences WSHPQFEKGGGSGGGSGGGSWSHPQFEKGASGEDYKDDDDK (SEQ ID NO: 30; core sequence of TAPe5), MAAASWSHPQFEKGGGSGGGSGGGSWSHPQFEK-GASGEDYKDDDDK (SEQ ID NO: 31; TAPe5), WSHPQFEKGGGSGGGSGGGS-WSHPQFEKGASGENLYFQGELDYKDHDGDYKDHDIDYKDDDDK (SEQ ID NO: 32; core sequence of TAPe6) or AAASWSHPQFEKGGGSGGGSGGGSWSHPQFEKGASG-ENLYFQGELDYKDHDGDYKDHDIDYKDDDDK (SEQ ID NO: 33, TAPe6) (the Strep-tags are in each case indicated by underlining (WSHPQFEK), the Flag-tags by italics (DYKDDDDK, DYKDHDGDYKDHDIDYKDDDDK) and the TEV-Cleavage sites (ENLYFQG; SEQ ID NO: 34) by bold-printed characters. The nucleic acid sequence of the affinity marker TAPe5 is given in SEQ ID No. 35.

The affinity marker according to the present invention can be produced for example by chemical synthesis in a known manner, e.g. by solid phase synthesis of linear peptide building blocks (SPPS) according to Merrifield using suitable protecting groups such as Fmoc. Individual components can also be joined together via peptide bonds. For this purpose it is possible to use e.g. amino acid coupling by activation of the carboxylic acid using for example 1-hydroxy-1H-benzotriazole (HOBt) and carbodiimide (e.g. EDC).

A further object of the present invention is a protein containing an affinity marker according to the invention. A protein according to the present invention is a polymer composed of amino acids, with the individual amino acids joined together by peptide bonds to form chains. The length of the amino acid chains can be more than 10000 amino acids, and is preferably more than 50 or more than 100 amino acids. The affinity marker that is bound to the protein to be purified is produced in a suitable manner by the techniques of genetic engineering using the corresponding nucleic acid and/or a suitable vector. These techniques are well known to a person skilled in the art and are described in more detail below. The protein contains both the FLAG-domain, optionally a cleavage site, for example a TEV or PreScission cleavage site, the Strep-domain, and the protein to be purified (protein sample).

The arrangement of the individual components in the protein with affinity marker, the underlying nucleic acid or the underlying vector can be as follows, for example:
FLAG-domain-protein-Strep-domain,
Strep-domain-protein- FLAG-domain,
FLAG-domain-Strep-domain -protein,
Strep-domain-FLAG-domain -protein,
Protein-FLAG-domain-Strep-domain or
Protein-Strep-domain-FLAG-domain

It may be necessary or desirable to separate the protein that is being purified from the affinity marker e.g. after purification, as the marker may affect e.g. the function or structure of the compound. Therefore the compound can be joined to the domain or domains via a cleavable linkage. The cleavable linkage is as defined above. If there are two cleavable linkages in the molecule or molecular complex, they are preferably different cleavable linkages, so as to allow e.g. selective cleavage of one of the two linkages. If only the FLAG-domain is bound directly to the protein, cleavage is accomplished preferably by means of enterokinase (as explained above), with the cleavage as a rule taking place according to the sequence DDDDK, e.g. in a FLAG-tag of DYKDDDDK.

The arrangement of the individual elements in the protein with affinity marker, the underlying nucleic acid or the underlying vector can be as follows, for example, and in these examples the order of the elements can be read from left to right or from right to left:
Strep-domain-optionally cleavage site 1-FLAG-domain-optionally cleavage site 2-Protein sample,
FLAG-domain-optionally cleavage site 1-Strep-domain-optionally cleavage site 2-Protein sample or
Strep-domain-optionally cleavage site 1-Protein sample-optionally cleavage site 2-FLAG-domain.

Cleavage sites 1 and 2 may be identical or preferably different. In the case of a cleavage site located C-terminally on the FLAG-tag, in particular it can be an enterokinase cleavage site, which cleaves according to the sequence DDDDK. Preferred cleavage sites, especially for cleavage sites between the two domains, are a TEV or PreScission cleavage site.

A further object of the present invention is a nucleic acid that codes for a protein containing the affinity marker according to the invention, provided the affinity marker is a pure protein. The nucleic acid may be, for example, an RNA or DNA. These can be used for example for production of the protein, labeled with the affinity marker that is to be purified. For this, the nucleic acid can be inserted in a cell, especially a eukaryotic cell, and expressed in the cell. Methods for the insertion and expression of nucleic acids in cells are generally familiar to a person skilled in the art. A nucleic acid containing or consisting of the sequence of SEQ ID NO: 35 is especially preferred.

Vectors are usually used for the production of proteins, especially of recombinant proteins. Accordingly, yet another object of the present invention is a vector containing a nucleic acid according to the invention. These vectors are then inserted in a target cell and the target cell is cultivated. With selection of a suitable vector, the target cell produces the desired protein. A great many vectors are known in the prior art. As a rule the vector is selected in relation to the target cell, in order to achieve expression that is as efficient as possible. In addition to the nucleic acid for the affinity marker optionally in combination with that for the protein of interest, the vector contains e.g. a suitable promoter, enhancer, selection marker and/or a suitable signal sequence, which for example causes the protein to be secreted into the medium, and optionally suitable cleavage sites for e.g. restriction enzymes. The constituents of vectors are familiar to a person skilled in the art, who will be able to select them in relation to the particular target cell. The restriction enzyme cleavage sites make it possible for nucleic acids that code for various proteins of interest to be incorporated in the vector and removed simply and quickly. Depending on what side the affinity marker is to be bound to the protein subsequently, they can be located 5′ or 3′ from the nucleic acid that codes for the affinity marker, or between the nucleic acid segments encoded by FLAG- and Strep-domain.

The protein contains the components FLAG- and Strep-domain plus the protein to be purified, each of which are as defined above and can also be arranged thus. Additionally there may be for example a spacer between the two domains, one or more cleavage sites or a signal sequence. The individual components are arranged in the protein as explained above. A person skilled in the art knows how to derive a nucleic acid sequence from a protein sequence, taking into account the genetic code. He also knows how to produce such a nucleic acid sequence using standard techniques of molecular biology. This can be accomplished for example by the use and combination of existing sequences using restriction enzymes. The nucleic acid suitably also contains further elements e.g. a promoter, enhancer, a transcription start and stop signal and a translation start signal.

The nucleic acid thus produced will then, optionally by means of a vector, be inserted and expressed in a target cell. Accordingly, a further object of the present invention is a cell containing a nucleic acid according to the invention or a vector according to the invention. The target cell can be any suitable cell, especially a eukaryotic cell, for example a fungal, plant or animal cell. Cell lines of these cells are also included. Preferably it is a mammalian cell, especially a human cell or cell line. Examples of such cells are HEK 293 cells, CHO cells, HeLa cells, CaCo cells or NIH 3T3 cells. Suitable vectors can be used for transfection of the cells. Examples of vectors are pBR322, the pUC series, pBluescript, pTZ, pSP and pGEM. The components of the nucleic acid or of the vector are selected in such a way that the nucleic acid is expressed and the target protein (affinity marker and protein of interest) is produced by the target cell. The cells are cultivated until a sufficient amount of target protein has been produced.

Then the protein can be isolated. If a sufficient amount of the target protein has been secreted into the medium, work can continue with this. Otherwise it may be necessary to disrupt the cells. This can be effected for example by lysis of the cells e.g. by means of ultrasound or hypotonic medium. To remove insoluble components, the sample obtained can for example be centrifuged, especially at 10000×g to 15000×g, and the supernatant obtained can be used further for the method of purification according to the invention (see below).

A further object of the present invention is a method for the purification of a protein expressed in a cell, especially a eukaryotic cell, preferably a mammalian cell, comprising the steps:
a) preparation of a protein according to the invention;
b) purifying the protein by means of affinity purification using the FLAG-domain; and
c) purifying the purified protein from step b) by means of affinity purification using the Strep-domain.

Alternatively, the following method can also be used:
a) preparation of a protein according to the invention;
b) purifying the protein by means of affinity purification using the Strep-domain; and
c) purifying the purified protein from step b) by means of affinity purification using the FLAG-domain.

The sample containing the molecule labeled with the affinity marker can be obtained as described above, for example using genetic engineering by expression in a cell, especially a eukaryotic cell.

The sample, obtained as described above, is purified by tandem affinity purification using the FLAG-domain and the Strep-domain. Affinity purification is a special form of adsorption purification, in which there are, on a carrier, groups (binding partners) with high affinity and therefore high binding strength to one of the two domains, so that these can be adsorbed preferentially and thus separated from other substances. Purification can be carried out either first via the FLAG-domain and then via the Strep-domain, or vice versa. Purification takes place by specific binding to a suitable binding partner. The binding partner is preferably bound to a solid phase. The solid phase can be usual carrier materials, for example Sepharose, Superflow, Macroprep, POROS 20 or POROS 50. Separation is then carried out for example chromatographically, e.g. by gravity, HPLC or FPLC. Alternatively, the binding partner can also be bound to beads, especially magnetic beads. After adding the beads to the sample, binding takes place between the particular domain and the corresponding binding partner. The suspension can then be centrifuged for example, so that the labeled molecule sediments with the bead, and other components remain in the supernatant, from where they can be removed. Alternatively, the suspension is separated utilizing the magnetic properties of the beads. In one embodiment, the suspension is applied to a column, which is located in a magnetic field. As the magnetic beads and the molecule bound to them are retained in the magnetic field, other constituents of the sample can be washed out in several washing operations. The molecule of interest can then for example be washed from the beads using a suitable elution buffer, or can be separated from the beads by enzymatic cleavage e.g. at the cleavage site between the FLAG-domain and the Strep-domain.

The first purification of the molecule (step b)) takes place by a) binding of the first domain (FLAG- or Strep-domain) to the respective binding partner (see above) in suitable conditions, b) optionally washing and c) detachment of the molecule from the binding partner. The latter can either be effected by altering the conditions, so that the changed conditions no longer permit binding between affinity marker and binding partner (e.g. alteration of the pH value or the ionic strength), or by separating the molecule from the domain bound to the binding partner. Separation can be effected by cleavage of the bond between molecule and binding partner, e.g. by chemical means or using specific enzymes, as was described in detail above. Alternatively, it is also possible to use specific competitors, which are added in excess.

This is followed by the second purification of the molecule (step c)) by a) binding of the second domain, i.e. the domain that was not used in the first purification step, to the respective binding partner in suitable conditions, b) optionally washing and c) detachment of the molecule from the binding partner, where these steps can be of the same form as those described for the first purification step.

In the case of the FLAG-domain, the binding partner is preferably a specific antibody. The antibody used can either be a polyclonal or preferably monoclonal antibody produced by methods known to a person skilled in the art or an antibody known from the prior art (see above). In this case elution of the bound protein can be carried out for example with the competitors such as synthetic FLAG- or 3×FLAG peptides (available from Sigma-Aldrich, Munich, Germany).

In the case of the Strep-domain, the binding partner is preferably Streptavidin or a related molecule such as core Streptavidin (Bayer et al., 1989, Biochem. J. 259: 369-376) or the Streptavidin muteins described in U.S. Pat. No. 6,103,493 or DE19641876. More preferably, the binding molecule is Strep-tactin. In this case elution of the bound protein can be effected for example with competitors such as biotin or biotin analogues such as desthiobiotin, iminobiotin, diaminobiotin, lipoic acid, HABA and/or DM-HABA. Furthermore, specific antibodies that are directed against the Strep-domain can also be used as the binding partner. The aforementioned Strep-tag binding partners are commercially available (IBA GmbH, Göttingen, Germany).

In one embodiment of the invention, the method of purification according to the invention can also be used in order to identify components (prey) which interact with a molecule labeled with the affinity marker (bait) and bind to it. Such bait/prey experiments are familiar to a person skilled in the art and are described in the examples.

In a preferred embodiment of the invention, the affinity marker is cleaved between step b) and step c). Cleavage takes place as described above. After step c), the affinity marker can be separated completely or partially from the protein. In a preferred embodiment the affinity marker is separated by incubation of the protein with the enzyme enterokinase.

Yet another object of the invention is the use of an affinity marker according to the invention for the purification of a protein from a cell, especially from a eukaryotic cell, preferably from a mammalian cell.

The present invention is additionally described by the following examples and drawings, which are not to be interpreted as limiting the scope of protection of the present invention.

### DESCRIPTION OF THE DRAWINGS

FIG. 1 shows the purification of B-Raf from HEK 293 cells.

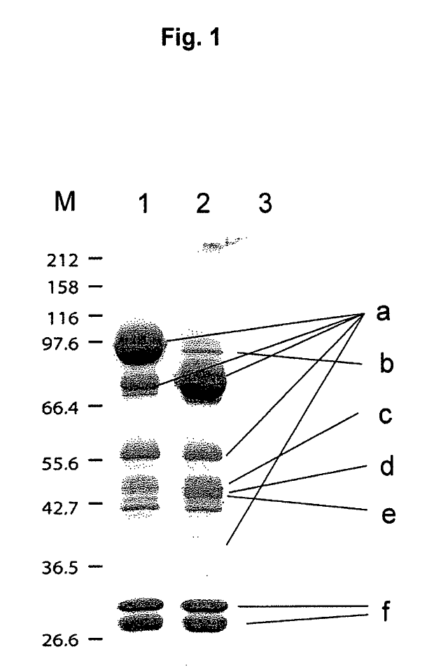

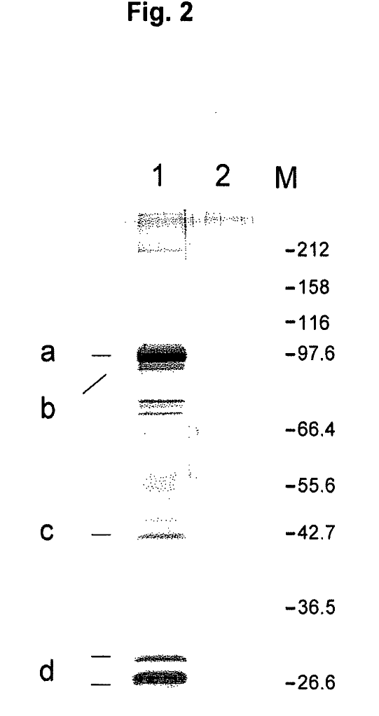

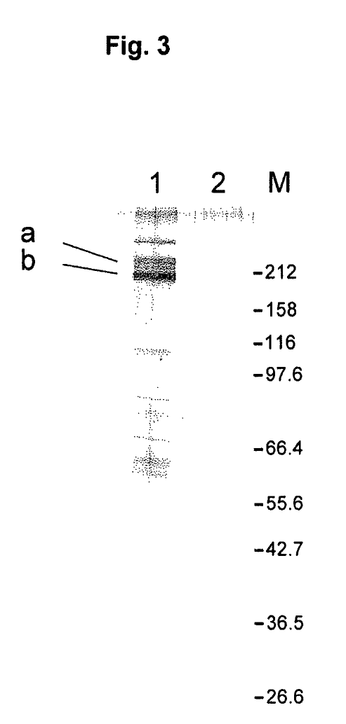

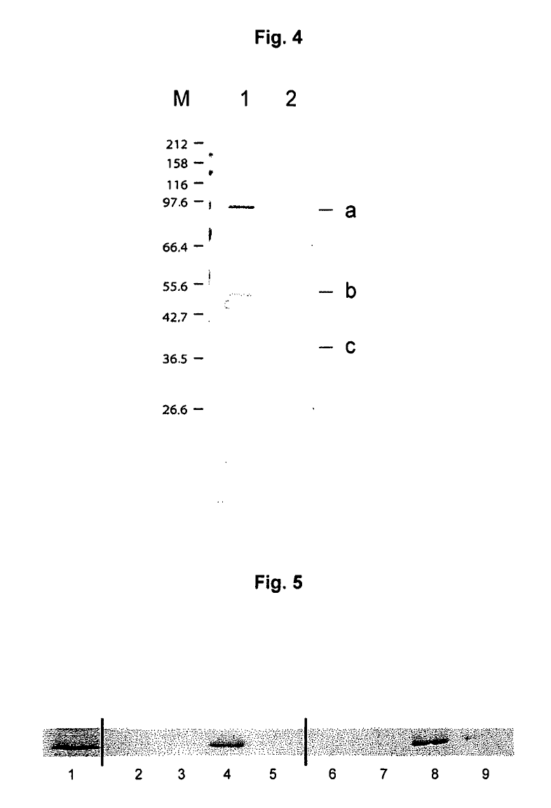

B-Raf was labeled with the marker TAPe5 and expressed in HEK 293 cells. On completion of cell lysis, a two-step purification of the TAPe5-labeled B-Raf was carried out. The precipitated proteins were separated on a 10% polyacrylamide gel. The diagram shows the resultant pattern of bands after staining the proteins with colloidal Coomassie Brilliant Blue G250. The co-purified proteins were identified by mass spectrometry after tryptic “in-gel”-proteolysis.

| M: molecular weight standard [kDa] | Proteins identified |
|---|---|
| 1: B-Raf TAPe5 | a: bait protein B-Raf and fragment |
| 2: Δ116 B-Raf TAPe5 | b: HSP90 |
| 3: vector control (TAPe5) | c: p50^{cdc37} |
| | d: MEK 2 |
| | e: MEK 1 |
| | f: [symbol] |

FIG. 2 illustrates the purification of B-Raf from Neuro2a cells.

On completion of cell lysis, a two-step purification of the TAPe5-labeled B-Raf was carried out. The precipitated proteins were separated on a 10% polyacrylamide gel. The diagram shows the resultant pattern of bands after silver staining of the proteins. The co-purified proteins were identified by mass spectrometry after tryptic “in-gel”-proteolysis.

| M: molecular weight standard [kDa] | Proteins noted |
|---|---|
| 1: B-Raf TAPe5 | a: B-Raf |
| 2: vector control (TAPe5) | b: HSP90 |
| | d: MEK |
| | f: 14-3-3 |

FIG. 3 shows the purification of RET9 from Neuro2a cells.

RET9 was labeled with the marker TAPe5 and expressed in Neuro2a cells. On completion of cell lysis, a two-step purification of the TAPe5-labeled B-Raf was carried out. The precipitated proteins were separated on a 10% polyacrylamide gel. The diagram shows the resultant pattern of bands after silver staining of the proteins.

| M: molecular weight standard [kDa] | Proteins identified |
|---|---|
| 1: RET9-TAPe5 | a, b: various glycosylation forms of |
| | RET9 |

FIG. 4 shows the purification of the kinase domain of LRRK2 from HEK293 cells as an example of a low-expressing bait protein.

The kinase domain of LRRK2 was labeled with the marker TAPe5 and expressed in HEK293 cells. On completion of cell lysis, a two-step purification of the TAPe5-labeled B-Raf was carried out. The precipitated proteins were separated on a 10% polyacrylamide gel. The diagram shows the resultant pattern of bands after staining the proteins with colloidal Coomassie Brilliant Blue G250.

| M: molecular weight standard [kDa] | Proteins identified |
|---|---|
| 1: TAPe5 labelled kinase domain | a: HSP90 |
| of LRRK2 | b: p50^{cdc37} |
| 2: vector control (TAPe5) | c: bait protein |

The co-purified proteins were identified by mass spectrometry after tryptic “in-gel”-proteolysis.

FIG. 5: shows the yields of the B-RAF-TAPe5 purification.

1% of the respective fractions was applied.

1: cell lysate
2: supernatant after Strep-purification
3: washing step
4: Strep-eluate)
5: stripped Streptactin-beads after elution
6: supernatant after FLAG-IP (2nd purification)
7: washing step
8: Flag-eluates (Flag-peptide
9: stripped Flag M2-Beads after elution

The affinity marker according to the invention with a combination of Tandem-StrepII and Flag-tag gave an efficiency of about 70% for the purification of B-Raf from HEK293 cells.

### EXAMPLE

### Tandem-Affinity Purification by Means of the TAPe5-Tag

1. Production of the constructs

The constructs were produced on the basis of the pcDNA3.0 vector (Invitrogen). The tags were cloned individually in the target vector in an oligonucleotide-based strategy. First, corresponding complementary oligonucleotide pairs were synthesized. The ends of the oligonucleotides were constructed so that after an annealing step (temperature-controlled condensation of complementary strands) overhangs formed, which were complementary to corresponding halves of the restriction endonucleases used, so that the resultant double-stranded fragment could be ligated in the prepared vector directly after annealing. The relevant techniques of molecular biology were carried out in accordance with standard protocols (Sambrook, J., Fritsch, E. F. and Maniatis, T., 1989, Molecular Cloning, Cold Spring Harbor Laboratory Press, 2nd edition).

2. Cultivation of the Cells

HEK293 and Neuro2a cells were cultivated in DMEM medium with 10% fetal calf serum (FBS). The conditions of cultivation comply with the standards (DSMZ, Braunschweig).

For transfection with construct-plasmids (TAPe5-pcDNA3) the cells were grown to a density of 50-80%. Transfection was carried out with Effectene (Qiagen, Hilden, Germany) in accordance with the manufacturer's instructions. The cells were incubated for 6 h with the transfection mix. Then the cells were kept in standard medium (DMEM, 10% FBS) for 48 h. Prior to the day before harvesting, the cells were starved in serum-free medium overnight.

3. Harvesting of the Cells

After removal of the medium, the cell culture plates (diameter 14 cm) were washed briefly with PBS, and liquid nitrogen was poured over them. After stimulation of the cells with growth factors, this quick freezing is necessary in order to prevent nonspecific phosphorylations.

4. Cell lysis

For cell lysis, 1 ml of lysis buffer per cell culture plate (50 mM Tris-HCl pH 7.4; 150 mM NaCl; 0.5-1% NP-40; 1× complete protease inhibitor cocktail (Roche); optionally (in the case of phosphorylated proteins) 1 mM phosphatase inhibitor orthovanadate) was added to the deep-frozen cell lawns and the cells were harvested by scraping. Then the samples were incubated for 30 min to 1 h at 4° C. in the overhead shaker and were centrifuged for 10 min at 10000×g (4° C.) to separate the nuclei. Undissolved constituents were removed from the supernatant by filtration through a 45μ filter unit (Millipore).

5. First Purification

About 20 μl Strep-Tactin Superflow Matrix (IBA) per ml of lysate (per 14 cm dish) was added to the clear lysates and they were incubated for 2 h at 4° C. in the overhead shaker. The beads were washed in spin-columns (GE-Healthcare) 3 to 5× with 500 μl 50 mM Tris-HCl pH 7.4, 150 mM NaCl with 1× complete protease inhibitor cocktail and 1 mM orthovanadate as phosphatase inhibitor. Elution was carried out twice with 2 to 3 bed-volumes (50-100 μl) of elution buffer (100 mM Tris-HCl pH 8.0; 150 mM NaCl, 1 mM EDTA; 2.5 mM desthiobiotin; optionally 1 mM orthovanadate). Prior to centrifugation (30 s; 2000×g) the beads were incubated on ice for 5 min.

6. Second Purification

An anti-FLAG-M2 matrix (Sigma-Aldrich) (about 10 μl per milliliter of initial lysate) was added to the first eluate and it was incubated for 2 h at 4° C. in the overhead shaker. At the end of the incubation time, the samples were transferred to MicroSpin-columns and washed 3 to 5× with 500 μl 50 mM Tris-HCl pH 7.4, 150 mM NaCl and 1 mM orthovanadate as phosphatase inhibitor. Elution was carried out using the FLAG-peptide (Sigma-Aldrich) at a concentration of 200 μg/ml in 50 mM Tris-HCl pH 7.4, 150 mM NaCl and 1 mM orthovanadate. 2 Bed-volumes of the elution solution are used, with incubation for 5 to 10 min at 4° C.

8. Gel Electrophoresis/Mass Spectrometry

Gel-electrophoretic separation was carried out using discontinuous SDS-polyacrylamide gel electrophoresis, modified according to Laemmli (Laemmli, 1970, Nature 227, 680-685). For visualization, the proteins were stained with silver in accordance with standard protocols compatible with mass spectrometry (Shevchenko et al., 1996, Analytical Chemistry, 68, 850-858). Protein identification was performed after in-gel proteolysis with trypsin on a MALDI (matrix assisted laser desorption and ionization)-TOF (time of flight) mass spectrometer by means of a peptide mass fingerprint (PMF) according to standard protocols (Mann, M., Hojrup, P. and Roepstorff, P., 1993, Biological Mass Spectrometry, 22, 338-345).

1. An affinity marker comprising
a FLAG-domain which contains at least one FLAG-tag, and
a Streptavidin-binding domain (Strep-domain) which contains at least two Strep-tags.

2. The affinity marker as claimed in claim 1, wherein the FLAG-domain contains or consists of at least 1, 2 or 3 FLAG-tags.

3. The affinity marker as claimed in claim 1, wherein the FLAG-tag contains or consists of at least one sequence selected from the group consisting of DYKD, DYKDDDDK, DFKDDDK, DYKAFDNL, DYKDHDG, MDFKDDDK, MDYKAFDNL, DYKDHDI and DYKDHDGDYKDHDIDYKDDDDK.

4. The affinity marker as claimed in claim 1, wherein the Strep-domain contains at least 3, 4, 5 or 6 Strep-tags.

5. The affinity marker as claimed in claim 1, wherein the Strep-tags contain an amino acid sequence selected from the group consisting of histidine-proline-glutamine, histidine-proline-asparagine, histidine-proline-methionine and WSHPQFEK.

6. The affinity marker as claimed in claim 1, wherein the Strep-domain contains exactly 2 Strep-tags and the FLAG-domain contains exactly 1 or exactly 3 FLAG-tags.

7. The affinity marker as claimed in claim 1, wherein there is a cleavable linkage between the FLAG-domain and the Strep-domain.

8. The affinity marker as claimed in claim 1, wherein the affinity marker contains or consists of an amino acid sequence selected from the group consisting of SEQ ID NO.30, 31, 32 and 33.

9. A protein containing an affinity marker as claimed in claim 1.

10. A nucleic acid coding for an affinity marker as claimed in claim 1.

11. A method for the purification of a protein expressed in a cell, comprising the steps:
a) preparation of a protein as claimed in claim 9;
b) purification of the protein by means of affinity purification using the FLAG-domain; and
c) purification of the purified protein from step b) by means of affinity purification using the Strep-domain.

12. A method for the purification of a protein expressed in a cell, comprising the steps:
a) preparation of a protein as claimed in claim 9;
b) purification of the protein by means of affinity purification using the Strep-domain; and
c) purification of the purified protein from step b) by means of affinity purification using the FLAG-domain.

13. The method as claimed in claim 11 or 12, wherein the cell is a eukaryotic cell.

14. The method as claimed in claim 11 or 12, wherein the purification using the FLAG-domain is carried out by means of an antibody to the FLAG-tag.

15. The method as claimed in claim 11 or 12, wherein the purification using the Strep-domain is carried out by means of Streptavidin and/or Strep-Tactin.


SEQUENCE LISTING (35 sequences)

SEQ ID NO: 1 - protein, 4 aa, Artificial sequence
  synthetic
DYKD

SEQ ID NO: 2 - protein, 8 aa, Artificial sequence
  synthetic
DYKDDDDK

SEQ ID NO: 3 - protein, 9 aa, Artificial sequence
  synthetic
MDYKDDDDK

SEQ ID NO: 4 - protein, 7 aa, Artificial sequence
  synthetic
DFKDDDK

SEQ ID NO: 5 - protein, 8 aa, Artificial sequence
  synthetic
DYKAFDNL

SEQ ID NO: 6 - protein, 7 aa, Artificial sequence
  synthetic
DYKDHDG

SEQ ID NO: 7 - protein, 9 aa, Artificial sequence
  synthetic
MDFKDDDDK

SEQ ID NO: 8 - protein, 9 aa, Artificial sequence
  synthetic
MDYKAFDNL

SEQ ID NO: 9 - protein, 7 aa, Artificial sequence
  synthetic
DYKDHDI

SEQ ID NO: 10 - protein, 5 aa, Artificial sequence
  synthetic
DYKDH

SEQ ID NO: 11 - protein, 5 aa, Artificial sequence
  synthetic
DYKDD

SEQ ID NO: 12 - protein, 6 aa, Artificial sequence
  synthetic
DYKDHD

SEQ ID NO: 13 - protein, 6 aa, Artificial sequence
  synthetic
DYKDDD

SEQ ID NO: 14 - protein, 22 aa, Artificial sequence
  synthetic
DYKDHDGDYKDHDIDYKDDDDK

SEQ ID NO: 15 - protein, 8 aa, artificial sequence
  synthetic
WSHPQFEK

SEQ ID NO: 16 - protein, 36 aa, Artificial sequence
  synthetic
  MISC_FEATURE (9)..(13): Xaa is any amino acid
  MISC_FEATURE (14)..(28): Xaa is any amino acid or is absent
WSHPQFEKXXXXXXXXXXXXXXXXXXXXWSHPQFEK

SEQ ID NO: 17 - protein, 21 aa, Artificial sequence
  synthetic
  MISC_FEATURE (9)..(13): Xaa is any amino acid
WSHPQFEKXXXXXWSHPQFEK

SEQ ID NO: 18 - protein, 24 aa, Artificial sequence
  synthetic
  MISC_FEATURE (9)..(16): Xaa is any amino acid
WSHPQFEKXXXXXXXXWSHPQFEK

SEQ ID NO: 19 - protein, 26 aa, Artificial sequence
  synthetic
  MISC_FEATURE (9)..(18): Xaa is any amino acid
WSHPQFEKXXXXXXXXXXWSHPQFEK

SEQ ID NO: 20 - protein, 28 aa, Artificial sequence
  synthetic
  MISC_FEATURE (9)..(20): Xaa is any amino acid
WSHPQFEKXXXXXXXXXXXXWSHPQFEK

SEQ ID NO: 21 - protein, 36 aa, Artificial sequence; identical to SEQ ID NO: 16
  synthetic
  MISC_FEATURE (9)..(28): Xaa is any amino acid

SEQ ID NO: 22 - protein, 24 aa, Artificial sequence
  synthetic
WSHPQFEKGGGGGGGGWSHPQFEK

SEQ ID NO: 23 - protein, 28 aa, Artificial sequence
  synthetic
WSHPQFEKGGGGGGGGGGGGWSHPQFEK

SEQ ID NO: 24 - protein, 20 aa, Artificial sequence
  synthetic
WSHPQFEKGGGSWSHPQFEK

SEQ ID NO: 25 - protein, 24 aa, Artificial sequence
  synthetic
WSHPQFEKGGGSGGGSWSHPQFEK

SEQ ID NO: 26 - protein, 28 aa, Artificial sequence
  synthetic
WSHPQFEKGGGSGGGSGGGSWSHPQFEK

SEQ ID NO: 27 - protein, 32 aa, Artificial sequence
  synthetic
WSHPQFEKGGGSGGGSGGGSGGGSWSHPQFEK

SEQ ID NO: 28 - protein, 36 aa, Artificial sequence
  synthetic
WSHPQFEKGGGSGGGSGGGSGGGSGGGSWSHPQFEK

SEQ ID NO: 29 - protein, 4 aa, Artificial sequence
  synthetic
DDDK

SEQ ID NO: 30 - protein, 41 aa, Artificial sequence
  synthetic
WSHPQFEKGGGSGGGSGGGSWSHPQFEKGASGEDYKDDDDK

SEQ ID NO: 31 - protein, 46 aa, Artificial sequence
  synthetic
MAAASWSHPQFEKGGGSGGGSGGGSWSHPQFEKGASGEDYKDDDDK

SEQ ID NO: 32 - protein, 63 aa, Artificial sequence
  synthetic
WSHPQFEKGGGSGGGSGGGSWSHPQFEKGASGENLYFQGELDYKDHDGDYKDHDIDYKDDDDK

SEQ ID NO: 33 - protein, 67 aa, Artificial sequence
  synthetic
AAASWSHPQFEKGGGSGGGSGGGSWSHPQFEKGASGENLYFQGELDYKDHDGDYKDHDIDYKDDDDK

SEQ ID NO: 34 - protein, 7 aa, Artificial sequence
  synthetic
ENLYFQG

SEQ ID NO: 35 - DNA, 141 bp, Artificial sequence
  synthetic
atggcggccgccagctggagccaccctcagttcgagaagggaggaggaagcggcggaggcagcggaggaggaagctggagccacccgcagttcgagaaaggagctagcggagaggattataaagatgatgatgataaatga